(19) United States
(12) Patent Application Publication
(10) Pub. No.: US 2024/0398751 A1
(43) Pub. Date: Dec. 5, 2024

(54) Methods of treating or reducing symptoms of stimulant dependency (addiction) via combination therapy with vaccine/antibodies and 5ht1/5ht2, SERT and glutamate allosteric modulators

(71) Applicant: David Alan Heldreth, JR., Bellevue, WA (US)

(72) Inventor: David Alan Heldreth, JR., Bellevue, WA (US)

(21) Appl. No.: 18/625,137

(22) Filed: Apr. 2, 2024

Related U.S. Application Data
(62) Division of application No. 17/667,147, filed on Feb. 8, 2022.
(60) Provisional application No. 63/207,183, filed on Feb. 10, 2021.

(51) Int. Cl. A61K 31/352; A61K 31/015; A61K 31/05; A61K 31/135; A61K 31/164; A61K 31/165; A61K 31/404; A61K 31/4045; A61K 31/473; A61K 31/4741; A61K 31/48; A61K 31/675; A61K 31/704; A61K 33/06; A61K 33/30; A61K 36/68; A61K 36/77

(52) U.S. Cl. CPC A61K 31/352; A61K 31/015; A61K 31/05; A61K 31/135; A61K 31/164; A61K 31/165; A61K 31/404; A61K 31/4045; A61K 31/473; A61K 31/4741; A61K 31/48; A61K 31/675; A61K 31/704; A61K 33/06; A61K 33/30; A61K 36/68; A61K 36/77

(57) ABSTRACT

The invention involves the use of formulations of allosteric modulators of primarily 5ht2a/5ht1a, opiate or SERT serotonin transporter, but also those of: 5ht1a/b/c/d, 5ht2a/b/c, 5ht3, 5ht4, 5ht7, dopamine, GLP, and other receptors/systems, in combination with phenethylamines, tryptamines, ibogaloids as well as vaccines, antibodies and other compounds; to treat stimulant dependency (addiction).

This patent (application) is a divisional patent filing which claims prior filing date/priority to patent, please reference patent title: Methods of use and formulations of allosteric modulators of the serotonin, dopamine and other receptor systems for medical, recreational, religious, research and other uses. Application Ser. No. 17/667,147—which claims prior filing date to a provisional patent. Please reference the provisional patent title: Psychedelic formulations for medical, recreational, religious, research and other uses. Application No. 63/207,183.

### FIELD OF THE INVENTION

The invention involves the use of formulations of allosteric modulators of primarily 5ht2a/5ht1a, glutamate or SERT serotonin transporter, but also those of: 5ht1a/b/c/d, 5ht2a/b/c, 5ht3, 5ht4, 5ht7, dopamine, GLP, and other receptors/systems, in combination with phenethylamines, tryptamines, ibogaloids as well as vaccines, antibodies and other compounds; to treat stimulant dependency (addiction).

### BACKGROUND OF THE INVENTION

People across the world are affected by stimulant dependency (addiction) and the serotonin system is believed to be involved in many if not all of these cases. Phenethylamines such as Mescaline, which is the active compound of many cacti, Psilocin/psilocybin containing mushrooms or fungi, have componds (primarily tryptamines) which cross the blood brain barrier and stimulate the 5-ht receptors. Phenethylamines such as Mescaline normescaline, the 2c series (2C-I/B/E*-NBOH NBOME etc) and MDMA, as well as lysergam ides and ergotamines and beta carbolines have activity on these systems. These compounds provide fast-acting and long-lasting changes to a person's illness.

However, there is variance in the effects these compounds have based on human genetics and related conditions. Allosteric binding occurs at a secondary binding site and not at the orthosteric or main agonist site and thus provides a way for additional compounds to be used in combination to provide ideal binding of receptors for individualized medicine and proper dosing. Allosteric binding affects the affinity and binding rate or activity of the receptor in a variety of ways including, but not limited to changing the shape of the receptor.

### SUMMARY OF THE INVENTION

The invention involves the use of formulations of allosteric modulators of primarily 5ht2a/5ht1a, glutamate or SERT serotonin transporter, but also those of: 5ht1a/b/c/d, 5ht2a/b/c, 5ht3, 5ht4, 5ht7, dopamine, GLP, and other receptors/systems, in combination with phenethylamines, tryptamines, ibogaloids as well as vaccines, antibodies and other compounds; to treat stimulant dependency (addiction).

### BRIEF DESCRIPTION OF THE DRAWINGS

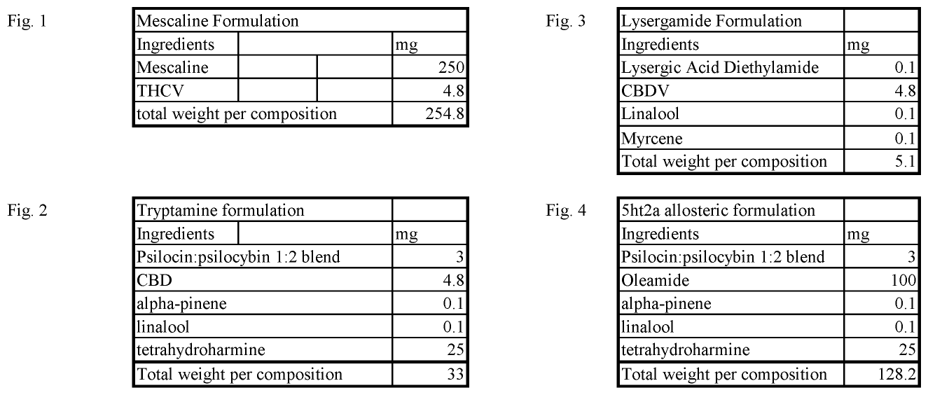

PAGE 1 of drawings show potential formulations of the compositions in FIG. 1, FIG. 2, FIG. 3 and FIG. 4. FIG. 1 shows a recipe for a mescaline and THCV composition to be taken orally, but may be used in other ways. FIG. 2 shows a recipe for psilocybin, psilocin, CBD, linalool, alpha-pinene and Tetrahydroharmine, composition to be taken orally, but may be used in other ways. FIG. 3 shows a recipe for LSD, CBDV, myrcene and linalool composition to be taken orally, but may be used in other ways. FIG. 4 shows a recipe for psilocybin, psilocin, CBD, linalool, alpha-pinene and Tetrahydroharmine additionally containing 5ht2a positive allosteric modulator oleamide, composition to be taken orally, but may be used in other ways.

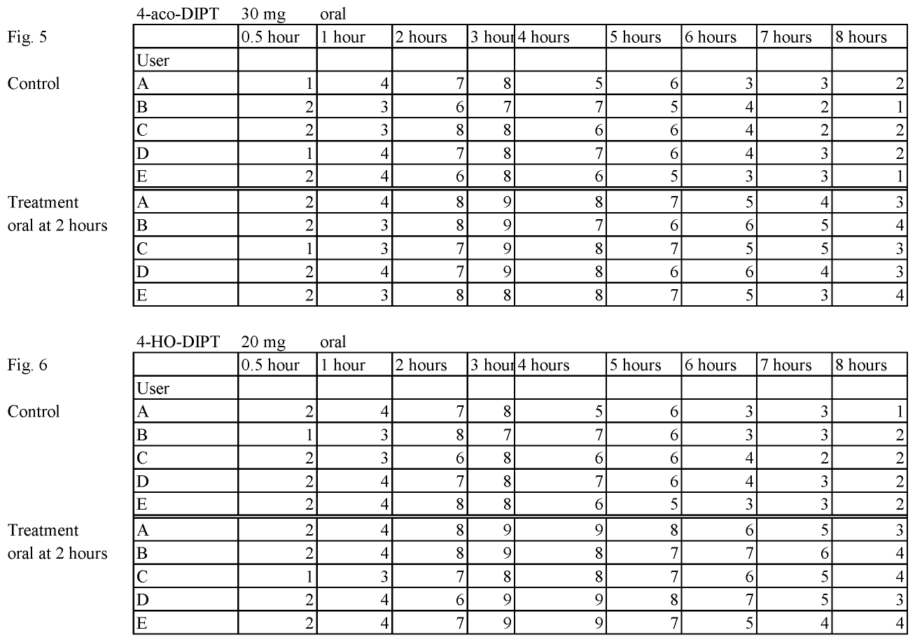

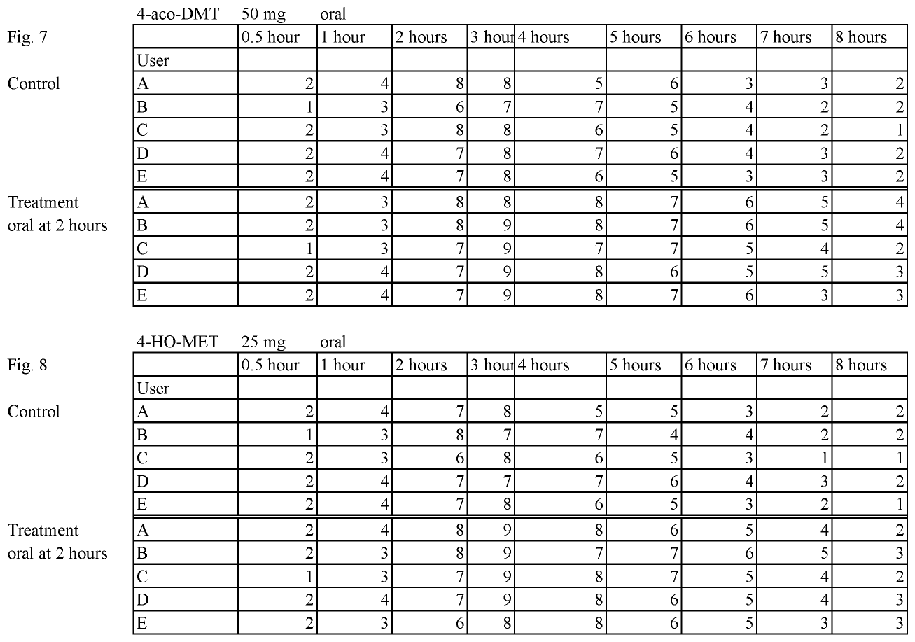

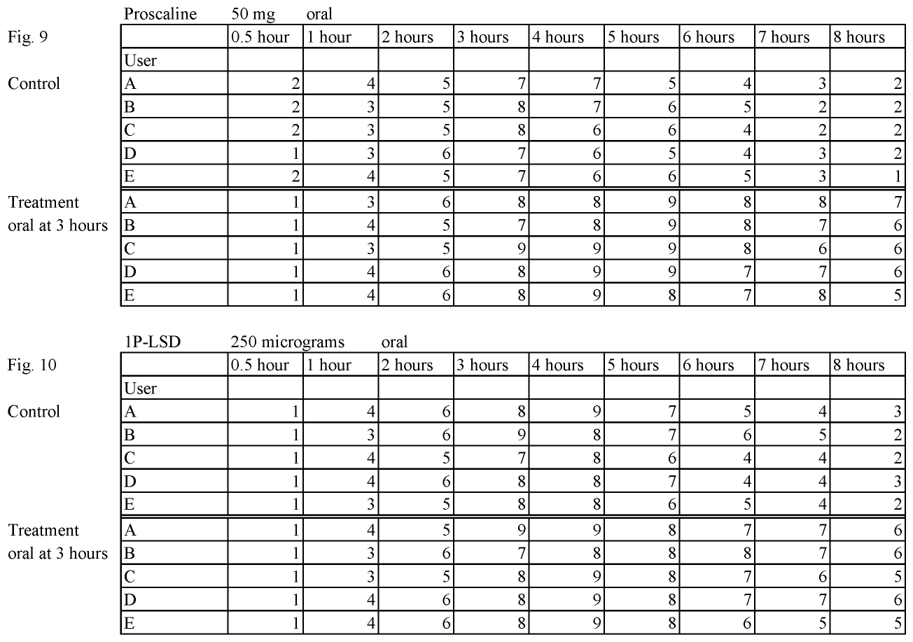

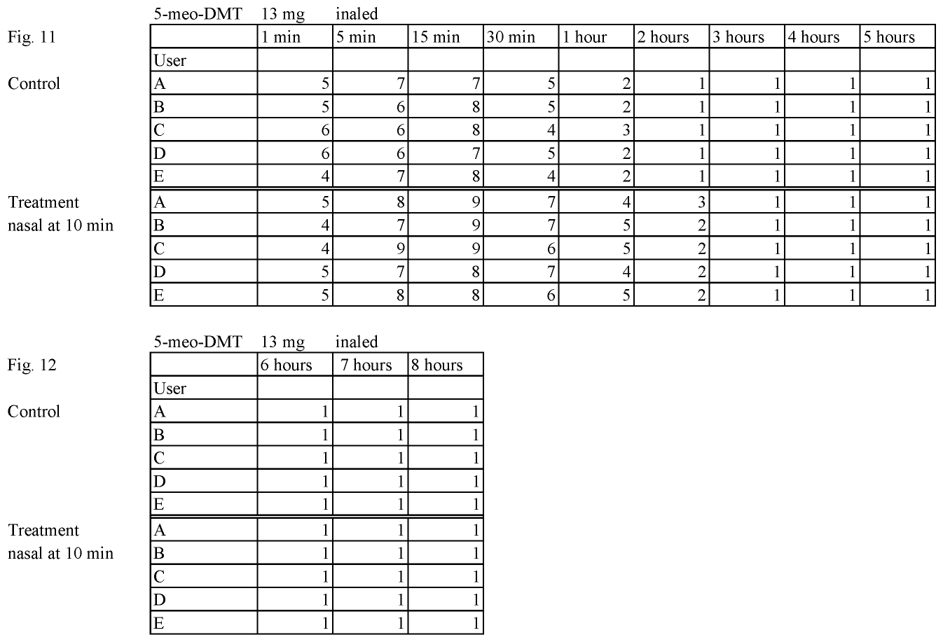

PAGES 2-5 of drawings show the results of a human experiment on the use of 4-aco-DIPT, 4-HO-DIPT, 4-aco-DMT, 4-ho-MET, proscaline, 1P-LSD and 5-MEO-DMT alone or with positive 5HT2A allosteric modulators. PAGE 2 shows FIG. 5: 4-aco-DIPT and FIG. 6: 4-HO-DIPT. PAGE 3 shows FIG. 7: 4-aco-DMT and FIG. 8: 4-ho-MET. PAGE 4 shows FIG. 9. Proscaline and FIG. 10: 1P-LSD. PAGE 5 shows FIG. 11 and FIG. 12: 5-MEO-DMT.

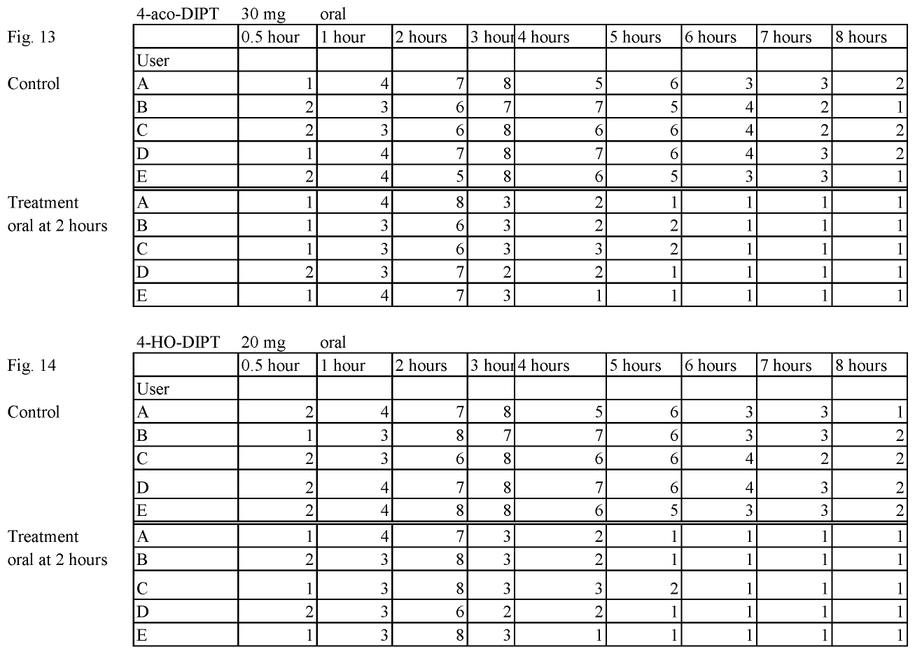

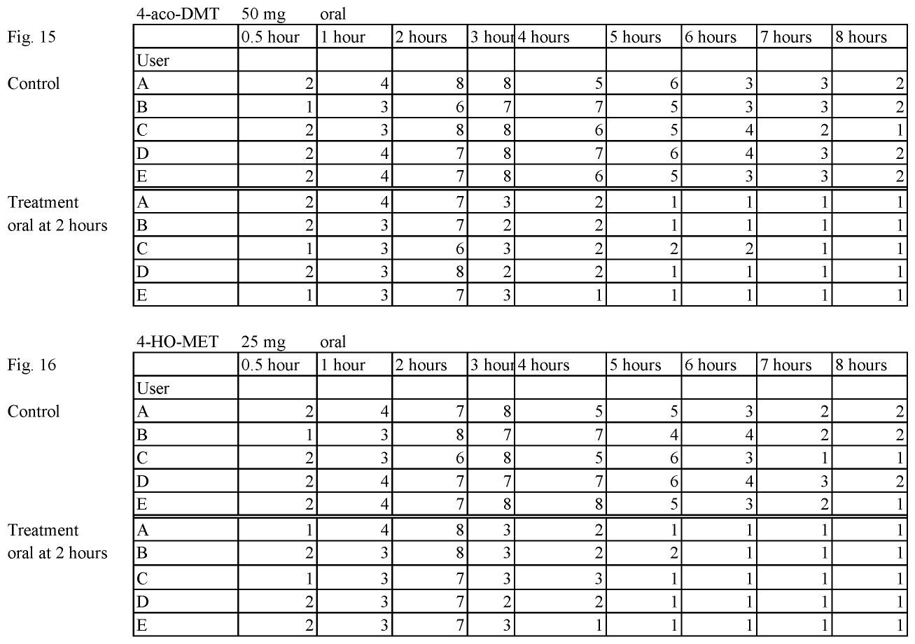

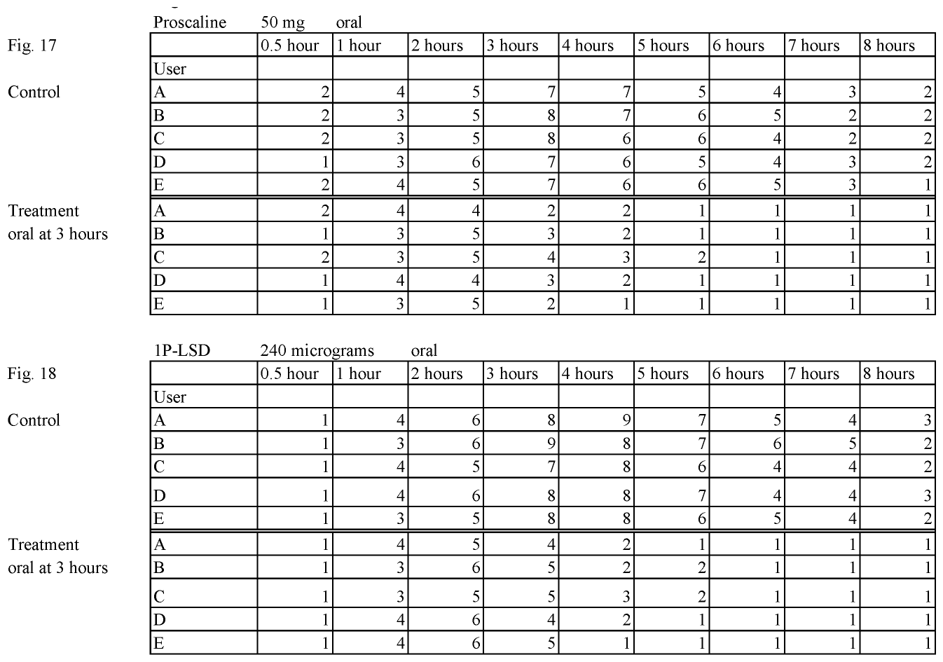

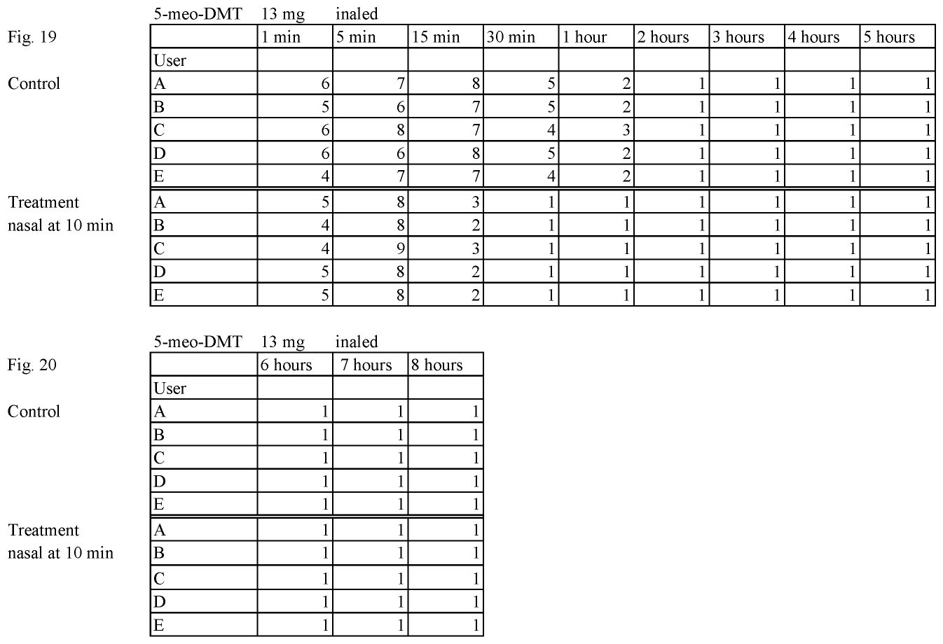

PAGES 6-9 of drawings show the results of a human experiment on the use of 4-aco-DIPT, 4-HO-DIPT, 4-aco-DMT, 4-ho-MET, proscaline, 1P-LSD and 5-MEO-DMT alone or with negative 5HT2A allosteric modulators. PAGE 6 shows FIG. 13: 4-aco-DIPT and FIG. 14: 4-HO-DIPT. PAGE 7 shows FIG. 15: 4-aco-DMT and FIG. 16: 4-ho-MET. PAGE 8 shows FIG. 17. Proscaline and FIG. 18: 1P-LSD. PAGE 9 shows FIG. 19 and FIG. 20: 5-MEO-DMT.

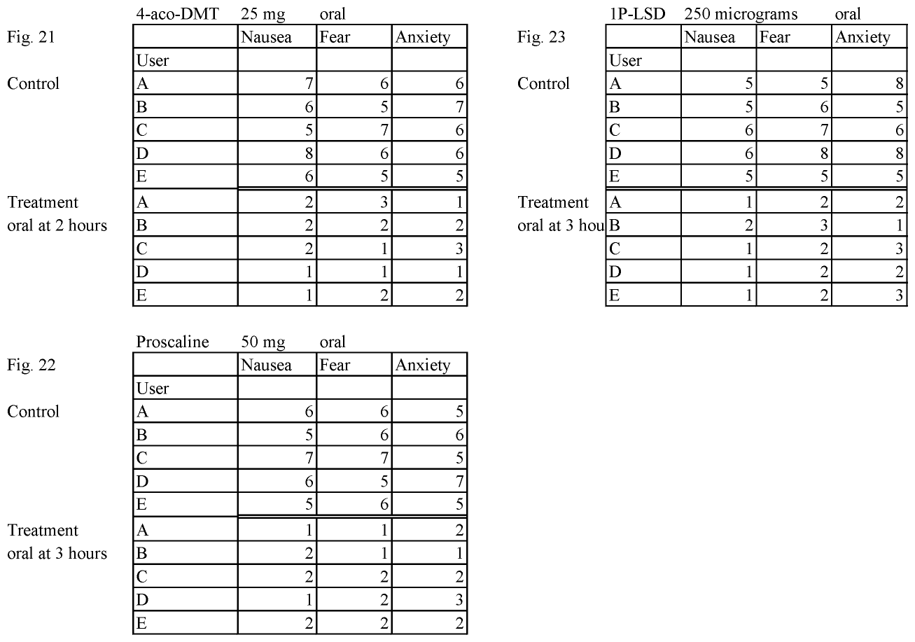

PAGE 10 shows the results of a human experiment on the use of 4-aco-DMT, proscaline or 1-P-LSD alone or with allosteric modulators to modulate fear, anxiety and nausea. The compounds used in addition to the psychedelics were: for Nausea: 5ht3 allosteric modulators: linalool, citral and ginger for nausea; for anxiety and fear: 5ht7 modulator zinc and 5ht1 modulator cannabidiol. FIG. 21: 4-aco-DMT. FIG. 22: Proscaline. FIG. 23: 1P-LSD.

### DRAWING SUMMARY

PAGE 1 of drawings show potential formulations of the compositions in FIG. 1, FIG. 2, FIG. 3 and FIG. 4. FIG. 1 shows a recipe for a mescaline and THCV composition to be taken orally, but may be used in other ways. FIG. 2 shows a recipe for psilocybin, psilocin, CBD, linalool, alpha-pinene and Tetrahydroharmine, composition to be taken orally, but may be used in other ways. FIG. 3 shows a recipe for LSD, CBDV, myrcene and linalool composition to be taken orally, but may be used in other ways. FIG. 4 shows a recipe for psilocybin, psilocin, CBD, linalool, alpha-pinene and Tetrahydroharmine additionally containing 5ht2a positive allosteric modulator oleamide, composition to be taken orally, but may be used in other ways.

PAGES 2-5 of drawings show the results of a human experiment on the use of 4-aco-DIPT, 4-HO-DIPT, 4-aco-DMT, 4-ho-MET, proscaline, 1P-LSD and 5-MEO-DMT alone or with positive 5HT2A allosteric modulators. PAGE 2 shows FIG. 5: 4-aco-DIPT and FIG. 6: 4-HO-DIPT. PAGE 3 shows FIG. 7: 4-aco-DMT and FIG. 8: 4-ho-MET. PAGE 4 shows FIG. 9. Proscaline and FIG. 10: 1P-LSD. PAGE 5 shows FIG. 11 and FIG. 12: 5-MEO-DMT.

PAGES 6-9 of drawings show the results of a human experiment on the use of 4-aco-DIPT, 4-HO-DIPT, 4-aco-DMT, 4-ho-MET, proscaline, 1P-LSD and 5-MEO-DMT alone or with negative 5HT2A allosteric modulators. PAGE 6 shows FIG. 13: 4-aco-DIPT and FIG. 14: 4-HO-DIPT. PAGE 7 shows FIG. 15: 4-aco-DMT and FIG. 16: 4-ho-MET. PAGE 8 shows FIG. 17. Proscaline and FIG. 18: 1P-LSD. PAGE 9 shows FIG. 19 and FIG. 20: 5-MEO-DMT.

PAGE 10 shows the results of a human experiment on the use of 4-aco-DMT, proscaline or 1-P-LSD alone or with allosteric modulators to modulate fear, anxiety and nausea. The compounds used in addition to the psychedelics were: for Nausea: 5ht3 allosteric modulators: linalool, citral and ginger for nausea; for anxiety and fear: 5ht7 modulator zinc and 5ht1 modulator cannabidiol. FIG. 21: 4-aco-DMT. FIG. 22: Proscaline. FIG. 23: 1P-LSD.

### DETAILED DESCRIPTION OF THE INVENTION

The invention involves the use of formulations of allosteric modulators of primarily 5ht2a/5ht1a, glutamate or SERT serotonin transporter, but also those of: 5ht1a/b/c/d, 5ht2a/b/c, 5ht3, 5ht4, 5ht7, dopamine, GLP, and other receptors/systems, in combination with phenethylamines, tryptamines, ibogaloids as well as vaccines, antibodies and other compounds; to treat stimulant dependency (addiction).

Allosteric modulation is the manipulation of a receptor at a site other than than normal binding site known as the orthosteric site. This is a less utilized method due to only recent discovery of its mechanisms as well as the need to identify these for each receptor. The use of allosteric modulators allows for precise alterations to the activity at the receptor and thus fine tuning of medical effects. Some embodiments utilize compounds which work as positive or negative allosteric modulators of the 5ht2a receptor or other 5ht systems. Allosteric modulation of 5ht2a also includes allosteric modulation through interaction with other receptor systems such as with hetomers or other such items.

In some embodiments the composition will include purified compounds which are either isolated or just purified. In other embodiments raw extracts or ground/processed biomass may be used. Some embodiments include excipients such as water, cyclodextrin, ethanol or other items off of the Food and Drug Administration authorized and approved excipient list.

Some embodiments will include utilizing nano technology, encapsulation, beta glucan particles, chitosan, yeast extract, surfactants, binders and other compounds to increase efficiency, availability, release lifespan, release speed among other parameters.

Some embodiments are used with vaccines, antibodies, cytokines, proteins, amino acids, DNA, RNA.

Potential compounds can be found below.

### Compound List

LSD, 1P-LSD, 1V-LSD, LSV, ALD-52, AL-LAD, and other LSD analogs, DBT, DET, DiPT, 5-MeO-α-MT, DMT, 2,α-DMT, α,N-DMT, DPT, EiPT, α-ET, ETH-LAD, Harmaline, Harmine, 4-HO-DBT, 4-HO-DET, 4-HO-DiPT, 4-HO-DMT, 5-HO-DMT, 4-HO-DPT, 4-HO-MET, 4-HO-MiPT, 4-HO-MPT, 4-HO-pyr-T, Ibogaine, LSD, MBT, 4,5-MDO-DiPT, 5,6-MDO-DiPT, 4,5-MDO-DMT, 5,6-MDO-DMT, 5,6-MDO-MiPT, 2-Me-DET, 2-Me-DMT, Melatonin, 5-MeO-DET, 5-MeO-DiPT, 5-MeO-DMT, 4-MeO-MiPT, 5-MeO-MiPT, 5,6-MeO-MiPT, 5-MeO-NMT, 5-MeO-pyr-T, 6-MeO-THH, 5-MeO-TMT, 5-MeS-DMT, MiPT, α-MT, NET, NMT, PRO-LAD, pyr-T, Tryptamine, Tetrahydroharmine, α,N,O-TMS, α,N,N-TMT⋅2,N,N-TMT⋅5,N,N-TMT⋅4-Acetoxy-DMT⋅4-Acetoxy-DET⋅4-Acetoxy-DIPT⋅4-HO-5-MeO-DMT⋅α-ET⋅α-MT⋅Baeocystin⋅Bufotenin⋅DBT⋅DET⋅DIPT⋅DMT⋅EiPT⋅PiPT⋅Ethocin⋅Ethocybin⋅Iprocin⋅4-HO-MET⋅4-HO-MiPT⋅MET⋅MIPT⋅5-Me-MIPT⋅5-MeO-α-ET⋅5-MeO-α-MT⋅5-MeO-DALT⋅5-MeO-DET⋅5-MeO-DIPT⋅5-MeO-DMT⋅5-MeO-DPT⋅5MeO-DPT⋅5-MeO-MET⋅5-MeO-MIPT⋅5-MeO-αN,N-TMT⋅5-MeO-2,N,N-TMT⋅Miprocin⋅Norbaeocystin⋅Psilocin⋅Psilocybin,4-HO-MALT, 4-Acetoxy-DET⋅4-Acetoxy-DIPT⋅4-Acetoxy-DMT⋅4-HO-DIPT⋅5-Bromo-DMT⋅5-Fluora-α-MT⋅5-MeO-α-ET⋅5-MeO-α-MT⋅5-MeO-DALT⋅5-MeO-DET⋅5-MeO-DIPT⋅5MeO-DMT⋅5-MeO-DPT⋅5-MeO-MIPT⋅α-ET⋅αMT⋅Baeocystin⋅Bufotenin⋅DET⋅DiPT⋅DMT⋅DPT⋅Ethocybin⋅EiPT⋅Ethocin⋅Ibogaine⋅Iprocin ⋅MET⋅MiPT⋅Miprocin⋅Melatonin⋅NMT Norbaeocystin⋅Normelatonin⋅PiPT⋅Psilocin⋅Psilocybin⋅Rizatriptan⋅Serotonin⋅Sumatriptan⋅Trypta mine⋅

Psilocybin (4-PO-HO-DMT), psilocin (4-HO-DMT), norpsilocin (ω-N-Methyl-4-hydroxytryptamine), baeocystin (4-PO-DMT), norbaeocystin (4-MeO-MIPT), Aeruginascin (N,N,N-trimethyl-4-phosphoryloxytryptamine), bufotenin (5-HO-DMT), 5-MEO-DMT, N,N-Dimethyltryptamine (N,N-DMT), 4-ACO-DMT, N-acetyl-4-hydroxytryptamine, 4-acetoxy-N-ethyl-N-methyltryptammonium (4-AcO-MET), 4-acetoxy-N,N-diallyltryptammonium (4-AcO-DALT) acid, 4-acetoxy-N-allyl-N-methyltryptammonium (4-AcO-MALT), N,N-dimethyl-N-n-propyltryptammonium (DMPT), N-allyl-N,N-dimethyltryptammonium (DMALT), 4-HO-TMT (4-hydroxy-N,N,N-trimethyltryptamine), N-methyltryptamine, 4-HO-NMT, 5-HO-NMT, 5-PO-HO-DMT, 4-PO-HO-NMT, 5-PO-HO-NMT, 4-HO-NMT, 4-HO-DMT, N-Methyl-4-phosphoryloxytryptamine, N,N-diethyl-tryptamine, 4-hydroxy-N,N-diethyltryptamine, 4-phosphoryloxy-N,N-diethyltryptamine, 5-HO-DET, 5-PO-DET, 4-PO-HO-DET, 5-PO-HO-DET, 5-PO-HO-DMT, 5-ACO-DMT, 4-ACO-NMT, 4-ACO-DET, 5-ACO-DET, 5-ACO-NMT, 4-hydroxy-6-methyl-L-tryptophan, 6-methyl psilocybin, 6-methyl psilocin, 6-methyl norpsilocin, 6-methyl baeocystin, 6-methyl norbaeocystin, 6-methyl Aeruginascin, 6-methyl bufotenin, 6-methylindole, 3-methylindole, 3-methyl baeocystin, 3-methyl norbaeocystin, 3-methyl Aeruginascin, 3-methyl bufotenin, 7-methylindole, 7-methyl baeocystin, 7-methyl norbaeocystin, 7-methyl Aeruginascin, 7-methyl bufotenin, 4-hydroxy-3-methyl-L-tryptophan, 4-hydroxy-7-methyl-L-tryptophan, 5-ht, gaba (gamma-Aminobutyric acid), serotonin, dopamine, epinephrine, oxytocin, tryptamine. 5-Bromo-DMT, 5,6-Dibromo-DMT, 4-aco-mpt, 4-aco-mipt, 4-aco-ept, 4-aco-dipt, 4-aco-dpt.

Iboga alkaloids such as, but not limited to: Coronaridine, Ibogamine, Voacangine, Ibogaine, conopharyngine, ibogaline, stemmadenine, noribogaine.

Cannabinoids including, but not limited to: endocannabinoids, phytocannabinoids, compounds which are ligands at CB1 or CB2 receptors such as, but not limited to: 2-AG, anandamide, CBD cannabidiol, THC (Tetrahydrocannabinol), Tetrahydrocannabivarin THCV, CBDV Cannabidivarin, tetrahydrocannabiphorol THCP, cannabidiphorol CBDP, hexyl CBD, THC acetates, CBD acetates, Cannabigerovarin CBGV, CBG cannabigerol, CBG acetates, heli-CBG, (CBC) cannabichromene, Cannabichromevarin CBCV, cannabinol CBN, beta caryophyllene, also butyl, hexyl, hepyl, octyl, deca, versions. Cannabinoids include all isomers including delta-8, delta-9, delta-10 and beyond such as for THC, but also including other cannabinoids.

Terpenes and terpenoids, such as, but not limited to: limonene, alpha-pinene, myrcene, linalool, terpinolene and all isomers of such compounds.

Phenethylamines including, but not limited to: mescaline, normescaline, 2c-i,2c-b, 2c-e. Includes all: phenethylamines in 2(×) series such as 2-CI including all NBOME, NBOH, and other analogs.

Beta carbolines and maoi inhibitors including, but not limited to: harmaline, tetraharmaline, norharmane, perlolyrine, tetrahydroharmine, harmane, harmine, barmol.

| | | |
|---|---|---|
| 1 | AEM | alpha-Ethyl-3,4,5-trimethoxy-PEA |
| 2 | AL | 4-Allyloxy-3,5-dimethoxy-PEA |
| 3 | ALEPH | 4-Methylthio-2,5-dimethoxy-A |
| 4 | ALEPH-2 | 4-Ethylthio-2,5-dimethoxy-A |
| 5 | ALEPH-4 | 4-Isopropylthio-2,5-dimethoxy-A |
| 6 | ALEPH-6 | 4-Phenylthio-2,5-dimethoxy-A |
| 7 | ALEPH-7 | 4-Propylthio-2,5-dimethoxy-A |
| 8 | ARIADNE | 2,5-Dimethoxy-alpha-ethyl-4-methyl-PEA |
| 9 | ASB | 3,4-Diethoxy-5-methoxy-PEA |
| 10 | B | 4-Butoxy-3,5-dimethoxy-PEA |
| 11 | BEATRICE | 2,5-Dimethoxy-4,N-dimethyl-A |
| 12 | BIS-TOM | 2,5-Bismethylthio-4-methyl-A |
| 13 | BOB | 4-Bromo-2,5, beta-trimethoxy-PEA |
| 14 | BOD | 2,5,beta-Trimethoxy-4-methyl-PEA |
| 15 | BOH | beta-Methoxy-3,4-methylenedioxy-PEA |
| 16 | BOHD | 2,5-Dimethoxy-beta-hydroxy-4-methyl-PEA |
| 17 | BOM | 3,4,5, beta-Tetram ethoxy-P E A |
| 18 | 4-Br-3,5-DMA | 4-Bromo-3,5-dimethoxy-A |
| 19 | 2-Br-4,5-MDA | 2-Bromo-4,5-methylenedioxy-A |
| 20 | 2C-B | 4-Bromo-2,5-dimethoxy-PEA |
| 21 | 3C-BZ | 4-Benzyloxy-3,5-dimethoxy-A |
| 22 | 2C-C | 4-Chloro-2,5-dimethoxy-PEA |
| 23 | 2C-D | 4-Methyl-2,5-dimethoxy-PEA |
| 24 | 2C-E | 4-Ethyl-2,5-dimethoxy-PEA |
| 25 | 3C-E | 4-Ethoxy-3,5-dimethoxy-A |
| 26 | 2C-F | 4-Fluoro-2,5-dimethoxy-PEA |
| 27 | 2C-G | 3,4-Dimethyl-2,5-dimethoxy-PEA |
| 28 | 2C-G-3 | 3,4-Trimethylene-2,5-dimethoxy-PEA |
| 29 | 2C-G-4 | 3,4-Tetramethylene-2,5-dimethoxy-PEA |
| 30 | 2C-G-5 | 3,4-Norbornyl-2,5-dimethoxy-PEA |
| 31 | 2C-G-N | 1,4-Dimethoxynaphthyl-2-ethylamine |
| 32 | 2C-H | 2,5-Dimethoxy-PEA |
| 33 | 2C-I | 4-lodo-2,5-dimethoxy-PEA |
| 34 | 2C-N | 4-Nitro-2,5-dimethoxy-PEA |
| 35 | 2C-O-4 | 4-Isopropoxy-2,5-dimethoxy-PEA |
| 36 | 2C-P | 4-Propyl-2,5-dimethoxy-PEA |
| 37 | CPM | 4-Cyclopropylmethoxy-3,5-dimethoxy-PEA |
| 38 | 2C-SE | 4-Methylseleno-2,5-dimethoxy-PEA |
| 39 | 2C-T | 4-Methylthio-2,5-dimethoxy-PEA |
| 40 | 2C-T-2 | 4-Ethylthio-2,5-dimethoxy-PEA |
| 41 | 2C-T-4 | 4-Isopropylthio-2,5-dimethoxy-PEA |
| 42 | psi-2C-T-4 | 4-Isopropylthio-2,6-dimethoxy-PEA |
| 43 | 2C-T-7 | 4-Propylthio-2,5-dimethoxy-PEA |
| 44 | 2C-T-8 | 4-Cyclopropylmethylthio-2,5-dimethoxy-PEA |
| 45 | 2C-T-9 | 4-(t)-Butylthio-2,5-dimethoxy-PEA |
| 46 | 2C-T-13 | 4-(2-Methoxyethylthio)-2,5-dimethoxy-PEA |
| 47 | 2C-T-15 | 4-Cyclopropylthio-2,5-dimethoxy-PEA |
| 48 | 2C-T-17 | 4-(s)-Butylthio-2,5-dimethoxy-PEA |
| 49 | 2C-T-21 | 4-(2-Fluoroethylthio)-2,5-dimethoxy-PEA |
| 50 | 4-D | 4-Trideuteromethyl-3,5-dimethoxy-PEA |
| 51 | beta-D | beta,beta-Dideutero-3,4,5-trimethoxy-PEA |
| 52 | DESOXY | 4-Methyl-3,5-Dimethoxy-PEA |
| 53 | 2,4-DMA | 2,4-Dimethoxy-A |
| 54 | 2,5-DMA | 2,5-Dimethoxy-A |
| 55 | 3,4-DMA | 3,4-Dimethoxy-A |
| 56 | DMCPA | 2-(2,5-Dimethoxy-4-methylphenyl)- |
| | | cyclopropylamine |
| 57 | DME | 3,4-Dimethoxy-beta-hydroxy-PEA |
| 58 | DMMDA | 2,5-Dimethoxy-3,4-methylenedioxy-A |
| 59 | DMMDA-2 | 2,3-Dimethoxy-4,5-methylenedioxy-A |
| 60 | DMPEA | 3,4-Dimethoxy-PEA |
| 61 | DOAM | 4-Amyl-2,5-dimethoxy-A |
| 62 | DOB | 4-Bromo-2,5-dimethoxy-A |
| 63 | DOBU | 4-Butyl-2,5-dimethoxy-A |
| 64 | DOC | 4-Chloro-2,5-dimethoxy-A |
| 65 | DOEF | 4-(2-Fluoroethyl)-2,5-dimethoxy-A |
| 66 | DOET | 4-Ethyl-2,5-dimethoxy-A |
| 67 | DOI | 4-lodo-2,5-dimethoxy-A |
| 68 | DOM (STP) | 4-Methyl -2,5-dimethoxy-A |
| 69 | psi-DOM | 4-Methyl -2,6-dimethoxy-A |
| 70 | DON | 4-Nitro-2,5-dimethoxy-A |
| 71 | DOPR | 4-Propyl-2,5-dimethoxy-A |
| 72 | E | 4-Ethoxy-3,5-dimethoxy-PEA |
| 73 | EEE | 2,4,5-Triethoxy-A |
| 74 | EEM | 2,4-Diethoxy-5-methoxy-A |
| 75 | EME | 2,5-Diethoxy-4-methoxy-A |
| 76 | EMM | 2-Ethoxy-4,5-dimethoxy-A |
| 77 | ETHYL-J | N, alpha-diethyl-3,4-methylenedioxy-PEA |
| 78 | ETHYL-K | N-Ethyl-alpha-propyl-3,4-methylenedioxy-PEA |
| 79 | F-2 | Benzofuran-2-methyl-5-methoxy- |
| | | 6-(2-aminopropane ) |
| 80 | F-22 | Benzofuran-2,2-dimethyl-5- |
| | | methoxy-6-(2-aminoprop ane) |
| 81 | FLEA | N-Hydroxy-N-methyl-3,4-methylenedioxy-A |
| 82 | G-3 | 3,4-Trimethylene-2,5-dimethoxy-A |
| 83 | G-4 | 3,4-Tetramethylene-2,5-dimethoxy-A |
| 84 | G-5 | 3,4-Norbornyl-2,5-dimethoxy-A |
| 85 | GANESHA | 3,4-Dimethyl-2,5-dimethoxy-A |
| 86 | G-N | 1,4-Dimethoxynaphthyl-2-isopropylamine |
| 87 | HOT-2 | 2,5-Dimethoxy-N-hydroxy-4-ethylthio-PEA |
| 88 | HOT-7 | 2,5-Dimethoxy-N-hydroxy-4-(n)-propylthio-PEA |
| 89 | HOT-17 | 2,5-Dimethoxy-N-hydroxy-4-(s)-butylthio-PEA |
| 90 | IDNNA | 2,5-Dimethoxy-N,N-dimethyl-4-iodo-A |
| 91 | IM | 2,3,4-Trimethoxy-PEA |
| 92 | IP | 3,5-Dimethoxy-4-isopropoxy-PEA |
| 93 | IRIS | 5-Ethoxy-2-methoxy-4-methyl-A |
| 94 | J | alpha-Ethyl-3,4-methylenedioxy-PEA |
| 95 | LOPHOPHINE | 3-Methoxy-4,5-methylenedioxy-PEA |
| 96 | M | 3,4,5-Trimethoxy-PEA |
| 97 | 4-MA | 4-Methoxy-A |
| 98 | MADAM-6 | 2,N-Dimethyl-4,5-methylenedioxy-A |
| 99 | MAL | 3,5-Dimethoxy-4-methallyloxy-PEA |
| 100 | MDA | 3,4-Methylenedioxy-A |
| 101 | MDAL | N-Allyl-3,4-methylenedioxy-A |
| 102 | MDBU | N-Butyl-3,4-methylenedioxy-A |
| 103 | MDBZ | N-Benzyl-3,4-methylenedioxy-A |
| 104 | MDCPM | N-Cyclopropylmethyl-3,4-methylenedioxy-A |
| 105 | MDDM | N,N-Dimethyl-3,4-methylenedioxy-A |
| 106 | MDE | N-Ethyl-3,4-methylenedioxy-A |
| 107 | MDHOET | N-(2-Hydroxyethyl)-3,4-methylenedioxy-A |
| 108 | MDIP | N-Isopropyl-3,4-methylenedioxy-A |
| 109 | MDMA | N-Methyl-3,4-methylenedioxy-A |
| 110 | MDMC | N-Methyl-3,4-ethylenedioxy-A |
| 111 | MDMEO | N-Methoxy-3,4-methylenedioxy-A |
| 112 | MDMEOET | N-(2-Methoxyethyl)-3,4-methylenedioxy-A |
| 113 | MDMP | alpha,alpha, N-Trimethyl-3,4- |
| | | methylenedioxy-PEA |
| 114 | MDOH | N-Hydroxy-3,4-methylenedioxy-A |
| 115 | MDPEA | 3,4-Methylenedioxy-PEA |
| 116 | MDPH | alpha,alpha-Dimethyl-3,4-methylenedioxy-PEA |
| 117 | MDPL | N-Propargyl-3,4-methylenedioxy-A |
| 118 | MDPR | N-Propyl-3,4-methylenedioxy-A |
| 119 | ME | 3,4-Dimethoxy-5-ethoxy-PEA |
| 120 | MEDA | 3-methoxy-4,5-Ethylenedioxy-A |
| | | [Erowid corrected] |
| 121 | MEE | 2-Methoxy-4,5-diethoxy-A |
| 122 | MEM | 2,5-Dimethoxy-4-ethoxy-A |
| 123 | MEPEA | 3-Methoxy-4-ethoxy-PEA |
| 124 | META-DOB | 5-Bromo-2,4-dimethoxy-A |
| 125 | META-DOT | 5-Methylthio-2,4-dimethoxy-A |
| 126 | METHYL-DMA | N-Methyl-2,5-dimethoxy-A |
| 127 | METHYL-DOB | 4-Bromo-2,5-dimethoxy-N-methyl-A |
| 128 | METHYL-J | N-Methyl-alpha-ethyl-3,4-methylenedioxy-PEA |
| 129 | METHYL-K | N-Methyl-alpha-propyl-3,4-methylenedioxy-PEA |
| 130 | METHYL-MA | N-Methyl-4-methoxy-A |
| 131 | METHYL- | N-Methyl-2-methoxy-4,5-methylenedioxy-A |
| | MMDA-2 | |
| 132 | MMDA | 3-Methoxy-4,5-methylenedioxy-A |
| 133 | MMDA-2 | 2-Methoxy-4,5-methylenedioxy-A |
| 134 | MMDA-3a | 2-Methoxy-3,4-methylenedioxy-A |
| 135 | MMDA-3b | 4-Methoxy-2,3-methylenedioxy-A |
| 136 | MME | 2,4-Dimethoxy-5-ethoxy-A |
| 137 | MP | 3,4-Dimethoxy-5-propoxy-PEA |
| 138 | MPM | 2,5-Dimethoxy-4-propoxy-A |
| 139 | ORTHO-DOT | 2-Methylthio-4,5-dimethoxy-A |
| 140 | P | 3,5-Dimethoxy-4-propoxy-PEA |
| 141 | PE | 3,5-Dimethoxy-4-phenethyloxy-PEA |
| 142 | PEA | PEA |
| 143 | PROPYNYL | 4-Propynyloxy-3,5-dimethoxy-PEA |
| 144 | SB | 3,5-Diethoxy-4-methoxy-PEA |
| 145 | TA | 2,3,4,5-Tetramethoxy-A |
| 146 | 3-TASB | 4-Ethoxy-3-ethylthio-5-methoxy-PEA |
| 172 | 5-TOM | 2-Methoxy-4-methyl-5-methylthio-A |
| 173 | TOMSO | 2-Methoxy-4-methyl-5-methylsulfinyl-A |
| 174 | TP | 4-Propylthio-3,5-dimethoxy-PEA |
| 175 | TRIS | 3,4,5-Triethoxy-PEA |
| 176 | 3-TSB | 3-Ethoxy-5-ethylthio-4-methoxy-PEA |
| 177 | 4-TSB | 3,5-Diethoxy-4-methylthio-PEA |
| 178 | 3-T-TRIS | 4,5-Diethoxy-3-ethylthio-PEA |
| 179 | 4-T-TRIS | 3,5-Diethoxy-4-ethylthio-PEA |

Additionally includes all: (4-acetoxy) (4-hydroxy) (dimethyl) (diethyl) (N-methyl-N-ethyl) (N-methyl) (N-methyl-N-isopropyl) (N,N-diisopropyl) variations of compounds in compound list.

Additionally includes all: functional group variants, fumerates, fumerics, idoines, hydrofumarates, deneutered or not, salts, acids, isomers, analogs, precursors, further metabolites of biosynthetic or synthetic pathways.

Also: Zinc, Magnesium, Sulfate, Carvelidol.

1. The method of treating or reducing symptoms (ovedose, addiction, etc) of stimulant use disorder (cocaine, amphetamine(s), methamphetamines, methylphenidate, and analogs or other stimulants); consisting of:
Administering to a human patient a therapeutic amount of a composition comprising: a vaccine or antibodies which targets one or a mixture of: cocaine, amphetamine(s), methamphetamines, methylphenidate, and analogs or other stimulants or their metabolites.
Administering to a human patient a therapeutic amount of a composition comprising:
a 5ht1a negative or 5ht2a positive, allosteric modulator or modulators, selected from: THCV (Tetrahydrocannabivarin), oleamide, linalool, limonene, tetrahydrocannabinol, cannabidiol, magnesium or zinc; AND
One item or a mixture of:
Mescaline, hordenine, anhalonidine, pellotine, proscaline (4-propoxy-3,5-dimethoxyphenethylamine or 4-propoxy-3,5-DMPEA), N-methylmescaline, N,N-Diformylmescaline, methylmescaline, normescaline, n-acetylmescaline, 2C-B, 2C-C, 2C-D, 2C-E, 2C-I, 2C-P, 2C-T, 2C-T2, 2C-T4, 2C-T7, 2C-T9, Trimethoxyamphetamine, or 3,4-Methylenedioxy methamphetamine; or other phenethylamine; Coronaridine, Ibogamine, Voacangine, Ibogaine, conopharyngine, ibogaline, stemmadenine, Noribogaine or other ibogaloid; a lysergide(s) such as, but not limited to LSD, LSZ, 1-PLSD, ALD-52, 1V-LSD, or other analogs; 4-ho-Met, 4-aco-Met, 5-MEO-Met, DipT, 4-ho-DiPT, 4-aco-DiPT, 5-MEO-DiPT, 4-aco-AMT, 4-ho-AMT, 5-MEO-AMT, 4-ho-MiPT, 4-aco-MiPT, 5-MEO-MiPT, 4-aco-DET, 4-ho-DET, 5-MEO-DET, or other tryptamine.
AND OPTIONALLY
Administering to a human patient a therapeutic amount of a composition comprising: a 5ht1a positive or 5ht2a negative, allosteric modulator or modulators selected from: palmitoleamide, 2,2-dimethyloleamide, N-oleoyl glycine, myristoleamide, 1-oleyl-2-acetylglycerol, anandamide, oleyl aldehyde, trifluoromethyl ketone, oleic acid, oleylpropanolamide alpha-pinene, cannabidiol, carvelidol or other allosteric compound of the receptors.

2. The method of claim 1 including testing/diagnosing the human patient such as, but not limited to: genetic testing, biomarkers.

3. The method of claim 1 including administering to a human patient a therapeutic amount of a composition comprising either:
harmine, hamaline, or other harmala alkaloids or a combination thereof;
OR
an allosteric modulator of GLP-1 or a dopamine receptor; OR
an allosteric modulator of the 5ht3, 5ht4, or 5ht7 receptors; OR
an Metabotropic Glutamate receptor allosteric modulator(s); OR
a serotonin transporter (SERT) allosteric modulator(s) or a mixture thereof such as: 3,4-methylenedioxy-methamphetamine (MDMA), citalopram, escitalopram, ocosahex-aenoic acid (DHA), vilazodone, (3-(1-(2-(1H-indol-3-yl)ethyl)piperidin-4-yl)-6-chloro-1H-indole), para-Trifluoromethyl-methcathinone, Butyrate.

4. The method of claim 1 including administering to a human patient a therapeutic amount of a composition comprising either: valencene, borneol, alpha-pinene, limonene, linalool, salivorin-A, citral, menthol, myrcene, other terpenes/terpenoids or a mixture thereof.

5. The method of claim 1 including administering to a human patient a therapeutic amount of: One item or a mixture of:
Mescaline, hordenine, anhalonidine, pellotine, proscaline (4-propoxy-3,5-dimethoxyphenethylamine or 4-propoxy-3,5-DMPEA), N-methylmescaline, N,N-Diformylmescaline, methylmescaline, normescaline, n-acetylmescaline, 2C-B, 2C-C, 2C-D, 2C-E, 2C-I, 2C-P, 2C-T, 2C-T2, 2C-T4, 2C-T7, 2C-T9, Trimethoxyamphetamine, or 3,4-Methylenedioxy methamphetamine; or other phenethylamine; Coronaridine, Ibogamine, Voacangine, Ibogaine, conopharyngine, ibogaline, stemmadenine, Noribogaine or other ibogaloid; a lysergide(s) such as, but not limited to LSD, LSZ, 1-PLSD, ALD-52, 1V-LSD, or other analogs; 4-ho-Met, 4-aco-Met, 5-MEO-Met, DipT, 4-ho-DiPT, 4-aco-DiPT, 5-MEO-DiPT, 4-aco-AMT, 4-ho-AMT, 5-MEO-AMT, 4-ho-MiPT, 4-aco-MiPT, 5-MEO-MiPT, 4-aco-DET, 4-ho-DET, 5-MEO-DET, or other tryptamine.

6. The method of claim 1 including administering to a human patient a therapeutic amount of a composition comprising a cannabinoid or cannabinoids or a mixture thereof.

7. The method of treating or reducing symptoms (ovedose, addiction, etc) of stimulant use disorder (cocaine, amphetamine(s), methamphetamines, methylphenidate, and analogs or other stimulants); consisting of:
Administering to a human patient a therapeutic amount of a composition comprising: a vaccine or antibodies which targets one or a mixture of: cocaine, amphetamine(s), methamphetamines, methylphenidate, and analogs or other stimulants or their metabolites.
Administering to a human patient a therapeutic amount of a composition comprising: serotonin transporter (SERT) allosteric modulator(s) or a mixture thereof such as: 3,4-methylenedioxy-methamphetamine (MDMA), citalopram, escitalopram, ocosahex-aenoic acid (DHA), vilazodone, (3-(1-(2-(1H-indol-3-yl)ethyl)piperidin-4-yl)-6-chloro-1H-indole), para-Trifluoromethyl-methcathinone, Butyrate.
AND OPTIONALLY
Administering to a human patient a therapeutic amount of a composition comprising either: valencene, borneol, alpha-pinene, limonene, linalool, salivorin-A, citral, menthol, myrcene, other terpenes/terpenoids or a mixture thereof.

8. The method of claim 7 including the administration to a human patient of a therapeutic amount of:
A 5ht2a negative allosteric modulator or modulators: such as, but not limited to zinc, alpha-pinene, 2,2-dimethyloleamide, palmitoleamide, N-oleoyl glycine, myristoleamide, 1-oleyl-2-acetylglycerol, anandamide;
OR
A 5ht1a positive allosteric modulator or modulators, such as, but not limited to: oleyl aldehyde, trifluoromethyl ketone, oleic acid, oleylpropanolamide.

9. The method of claim 7 including the administration to a human patient of a therapeutic amount of:
a 5ht2a positive allosteric modulator or modulators, such as, but not limited to: oleamide, linalool, limonene, tetrahydrocannabinol;
AND/OR
a 5ht1a negative allosteric modulator or modulators, such as, but not limited to: THCV or zinc.

10. The method of claim 7 including testing/diagnosing the human patient such as, but not limited to: genetic testing, biomarkers.

11. The method of claim 7 including administering to a human patient a therapeutic amount of a composition comprising either:
harmine, hamaline, or other harmala alkaloids or a combination thereof;
OR
an allosteric modulator of GLP-1 or a dopamine receptor; OR
an allosteric modulator of the 5ht3, 5ht4, or 5ht7 receptors; OR
an Metabotropic Glutamate receptor allosteric modulator(s).

12. The method of claim 7 including administering to a human patient a therapeutic amount of a composition comprising a cannabinoid or cannabinoids or a mixture thereof.

13. The method of claim 7 including administering to a human patient a therapeutic amount of: One item or a mixture of:
mescaline, hordenine, anhalonidine, pellotine, proscaline (4-propoxy-3,5-dimethoxyphenethylamine or 4-propoxy-3,5-DMPEA), N-methylmescaline, N,N-Diformylmescaline, methylmescaline, normescaline, n-acetylmescaline, 2C-B, 2C-C, 2C-D, 2C-E, 2C-I, 2C-P, 2C-T, 2C-T2, 2C-T4, 2C-T7, 2C-T9, Trimethoxyamphetamine, or 3,4-Methylenedioxy methamphetamine; or other phenethylamine;
Coronaridine, Ibogamine, Voacangine, Ibogaine, conopharyngine, ibogaline, stemmadenine, Noribogaine or
lysergides such as, but not limited to LSD, LSZ, 1-PLSD, ALD-52, 1V-LSD, or other analogs.
4-ho-Met, 4-aco-Met, 5-MEO-Met, DipT, 4-ho-DiPT, 4-aco-DiPT, 5-MEO-DiPT, 4-aco-AMT, 4-ho-AMT, 5-MEO-AMT, 4-ho-MiPT, 4-aco-MiPT, 5-MEO-MiPT, 4-aco-DET, 4-ho-DET, 5-MEO-DET, or other tryptamine.

14. The method of treating or reducing symptoms (ovedose, addiction, etc) of stimulant use disorder (cocaine, amphetamine(s), methamphetamines, methylphenidate, and analogs or other stimulants); consisting of:
Administering to a human patient a therapeutic amount of a composition comprising: a vaccine or antibodies which targets one or a mixture of: cocaine, amphetamine(s), methamphetamines, methylphenidate, and analogs or other stimulants or their metabolites.
Administering to a human patient a therapeutic amount of a composition comprising: Metabotropic Glutamate Receptor allosteric modulator(s);
AND OPTIONALLY
Administering to a human patient a therapeutic amount of a composition comprising either: valencene, borneol, alpha-pinene, limonene, linalool, salivorin-A, citral, menthol, myrcene, other terpenes/terpenoids or a mixture thereof.

15. The method of claim 14 including the administration to a human patient of a therapeutic amount of:
A 5ht2a negative allosteric modulator or modulators: such as, but not limited to zinc, alpha-pinene, 2,2-dimethyloleamide, palmitoleamide, N-oleoyl glycine, myristoleamide, 1-oleyl-2-acetylglycerol, anandamide;
OR
A 5ht1a positive allosteric modulator or modulators, such as, but not limited to: oleyl aldehyde, trifluoromethyl ketone, oleic acid, oleylpropanolamide.

16. The method of claim 14 including the administration to a human patient of a therapeutic amount of:
a 5ht2a positive allosteric modulator or modulators, such as, but not limited to: oleamide, linalool, limonene, tetrahydrocannabinol;
AND/OR
a 5ht1a negative allosteric modulator or modulators, such as, but not limited to: THCV or zinc.

17. The method of claim 14 including testing/diagnosing the human patient such as, but not limited to: genetic testing, biomarkers.

18. The method of claim 14 including administering to a human patient a therapeutic amount of a composition comprising either:
harmine, hamaline, or other harmala alkaloids or a combination thereof;
OR
an allosteric modulator of GLP-1 or a dopamine receptor; OR
an allosteric modulator of the 5ht3, 5ht4, or 5ht7 receptors; OR
a serotonin transporter (SERT) allosteric modulator(s) or a mixture thereof such as: 3,4-methylenedioxy-methamphetamine (MDMA), citalopram, escitalopram, ocosahex-aenoic acid (DHA), vilazodone, (3-(1-(2-(1H-indol-3-yl)ethyl)piperidin-4-yl)-6-chloro-1H-indole), para-Trifluoromethyl-methcathinone, Butyrate.

19. The method of claim 14 including administering to a human patient a therapeutic amount of a composition comprising a cannabinoid or cannabinoids or a mixture thereof.

20. The method of claim 14 including administering to a human patient a therapeutic amount of: One item or a mixture of:
mescaline, hordenine, anhalonidine, pellotine, proscaline (4-propoxy-3,5-dimethoxyphenethylamine or 4-propoxy-3,5-DMPEA), N-methylmescaline, N,N-Diformylmescaline, methylmescaline, normescaline, n-acetylmescaline, 2C-B, 2C-C, 2C-D, 2C-E, 2C-I, 2C-P, 2C-T, 2C-T2, 2C-T4, 2C-T7, 2C-T9, Trimethoxyamphetamine, or 3,4-Methylenedioxy methamphetamine; or other phenethylamine;
Coronaridine, Ibogamine, Voacangine, Ibogaine, conopharyngine, ibogaline, stemmadenine, Noribogaine or
lysergides such as, but not limited to LSD, LSZ, 1-PLSD, ALD-52, 1V-LSD, or other analogs.
4-ho-Met, 4-aco-Met, 5-MEO-Met, DipT, 4-ho-DiPT, 4-aco-DiPT, 5-MEO-DiPT, 4-aco-AMT, 4-ho-AMT, 5-MEO-AMT, 4-ho-MiPT, 4-aco-MiPT, 5-MEO-MiPT, 4-aco-DET, 4-ho-DET, 5-MEO-DET, or other tryptamine.
